(19) United States
(12) Patent Application Publication
(10) Pub. No.: US 2022/0031625 A1
(43) Pub. Date: Feb. 3, 2022

(54) OPHTHALMIC COMPOSITIONS FOR DELIVERING MEIBUM-LIKE MATERIALS

(71) Applicant: Altaire Pharmaceuticals, Inc., Aquebogue, NY (US)

(72) Inventor: Assad Sawaya, Baiting Hollow, NY (US)

(21) Appl. No.: 17/389,639

(22) Filed: Jul. 30, 2021

Related U.S. Application Data
(60) Provisional application No. 63/059,261, filed on Jul. 31, 2020.

(51) Int. Cl. A61K 9/50; A61K 9/00; A61K 47/28; A61K 47/14; A61K 38/13; A61K 31/573; A61K 31/192; A61K 31/196; A61K 31/7048; A61K 31/137; A61K 31/138; A61K 31/7036; A61K 31/498; A61K 31/18; A61K 9/06

(52) U.S. Cl. CPC A61K 9/5031; A61K 9/06; A61K 47/28; A61K 47/14; A61K 38/13; A61K 31/573; A61K 31/192; A61K 31/196; A61K 31/7048; A61K 31/137; A61K 31/138; A61K 31/7036; A61K 31/498; A61K 31/18; A61K 9/0048

(57) ABSTRACT

Artificial tear formulations include nanoparticles having a nonpolar meibum-like material encapsulated by a polyoxyethylene castor oil derivative and exhibits a clear appearance. The meibum-like material may include a cholesteryl ester and/or a wax ester. The polyoxyethylene castor oil derivative may be polyoxyl 35 castor oil. The formulation, which may be a solution suitable for eye drops or a gel/ointment, may have a pH of 6.0-8.0, and an osmolality of from 250 mOsm/kg to 400 mOsm/kg.

### CROSS-REFERENCE TO RELATED APPLICATIONS

This application claims the benefit of, and priority to, U.S. Provisional Patent Application No. 63/059,261, filed on Jul. 31, 2020, the entire contents of which are hereby incorporated herein by reference.

### TECHNICAL FIELD

This disclosure relates to artificial tear compositions useful, for example, in treating dry eye disease and meibomian gland dysfunction (MGD). In particular, this disclosure relates to clear ophthalmic microemulsions containing nonpolar lipids and wax esters such as, for example, those present in the lipid layer of human tear film and meibum.

### BACKGROUND

The human tear film consists of three layers: a mucin layer immediately on the top of the ocular surface; an intermediate aqueous layer which is the main body of tear film; and an outmost lipid layer which reduces evaporation of water from the aqueous layer to the atmosphere. Dry eye patients typically feel inadequate water in the tear film, mostly due to accelerated evaporation of water to the atmosphere when the protective lipid layer is compromised. For severe dry eyes, some epithelial cells on the ocular surface can be exposed to the atmosphere and become desiccated and die. Therefore, additional blinks of the eyes are needed to replenish the tear film and to compensate for the demand of water. Normal tear break-up time (“TBUT”) is about ten seconds. A TBUT shorter than ten seconds indicates a dry eye condition. An immediate treatment for dry eye symptoms is to provide an excess of an artificial tear composition which is mostly aqueous solution in order to alleviate the shortage of water in the eye.

It has been reported that meibomian gland dysfunction (“MGD”) is the root cause for most of the population with dry eye disease because meibomian glands play the role of secreting the lipid layer materials. Meibomian glands are a group of sebaceous glands located on the inside of the eyelids, producing oily lipid substances called meibum, inhibiting evaporation of the tear film. A deficient meibum secretion will lead to insufficient stabilization of the tear film and eventually dry eye disease. MGD is associated with various conditions, such as the obstruction of the meibomian gland channels and/or incorrect lipid/oil compositions of the human meibum.

Although most of the artificial tears currently on the market are successful in restoring/replenishing the aqueous portion of the tear film, a satisfactory artificial tear product should not only address the aqueous phase, but also the oil phase or lipids of the tear film. Nonpolar oils such as mineral oil and castor oil have been used as emollients in ophthalmic solutions in recent years in an effort to support both the aqueous layer and lipid layer in tear films. Although the majority of nonpolar lipids in meibum are cholesteryl esters and wax esters, these compounds have not been used in currently marketed OTC/Rx artificial tears.

In addition, the particle sizes of artificial tears currently on the market are typically a few hundred nanometers, which leads to haziness and cloudiness, yielding an unfavorable white, milky visual appearance. Instillation of such products into eyes will result in temporary blurring of vision and lead to the limitation of use in circumstances when consistent, clear vision is important and necessary (e.g., during driving).

It would be desirable to design a clear eye drop product that addresses both restoration of the aqueous layer and the lipid layer of the tear film.

### SUMMARY

Ophthalmic formulations in accordance with the present disclosure include nonpolar meibum-like materials, such as, for example, cholesteryl esters and wax esters, encapsulated in nanoparticle micelles formed by polyoxyethylene castor oil derivatives, such as polyoxyl 35 castor oil, to provide a clear aqueous solution/gel formulation. The nanoparticles accommodate nonpolar materials in the nanometer regime. By adjusting the polarity ratio of nonpolar meibum-like materials and the polyoxyethylene castor oil derivatives (e.g., polyoxyl 35 castor oil), various formulations with encapsulated meibum-like materials can be generated. These oil-in-water microemulsion preparations are stable and have a particle size from about 5 nm to about 100 nm, providing a clear solution appearance and the capability of preparing formulations ranging from low-viscosity eye drops to high-viscosity aqueous gels/ointments. In other aspects, the present disclosure relates to the use of the disclosed formulations to treat MGD and dry eye disease. The formulations mimic the chemical composition of human tear film and lipidome, and have nonpolar lipids encapsulated in the nanometer regime.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the terms “treat,” “treated,” or “treating” mean both therapeutic treatment and prophylactic or preventative measures wherein the object is to prevent or slow down (lessen) an undesired physiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects.

### BRIEF DESCRIPTION OF THE DRAWINGS

A more complete understanding of the presently disclosed concepts and illustrative embodiments may be acquired by referring to the following description, taken in conjunction with the figures of the accompanying drawings wherein:

FIG. 1 shows a Gaussian distribution of the nanoparticles in compositions in accordance with the present disclosure containing cholesteryl linolenate, oleyl linoleate, and 20% polyoxyl 35 castor oil; the microemulsion system is a clear buffered solution with mean particle size=14.3 nm (range 2 nm to 80 nm); and

FIG. 2 shows a Gaussian distribution of the nanoparticles in a microemulsion compositions in accordance with the present disclosure containing 0.1% (w/v) cyclosporine. The mean size of the nanoparticles is 20.9 nm and the size range of the nanoparticles is from 5 nm to 50 nm.

### DETAILED DESCRIPTION

In illustrative embodiments described herein, clear ophthalmic compositions include nanoparticles of nonpolar meibum-like materials, such as, for example, cholesteryl/sterol esters and wax esters, encapsulated with polyoxyethylene castor oil derivatives.

Polyoxyethylene castor oil derivatives are produced by reacting varying amounts of ethylene oxide with castor oil under raised pressure and temperature in the presence of a catalyst. Different polyethoxylated castor oils can be produced by controlling the molar ratio of ethylene oxide relative to castor oil. Polyoxyethylene castor oil derivatives (sometimes also called polyoxyl castor oil, polyoxyl n castor oil, polyethylene glycol castor oil, castor oil ethoxylates, or polyethoxylated castor oil) are complex mixtures of various hydrophobic and hydrophilic components. In polyethoxylated castor oil, the hydrophobic constituents comprise about 80% of the total mixture, the main component being glycerol polyethylene glycol ricinoleate. Other hydrophobic constituents include fatty acid esters of polyethylene glycol along with some unchanged castor oil. The hydrophilic part consists of polyethylene glycols and glycerol ethoxylates.

When referred to as “polyoxyl n castor oil”, the number (n) associated with the name of the substance represents the average number of oxyethylene units in the compound. Polyoxyl n castor oil where n=30 to 40 is a mixture of tri-ricinoleate esters of ethoxylated glycerol with small amounts of polyethyleneglycol (macrogol) ricinoleate and the corresponding free glycols. Polyoxyl n hydrogenated castor oil where n=40 to 60 is a mixture of tri-hydroxystearate esters of ethoxylated glycerol with small amounts of macrogol tri-hydroxystearate and the corresponding free glycols. Polyoxyl castor oils and polyoxyl hydrogenated castor oils are nonionic surfactants.

Suitable polyoxyethylene castor oil derivatives include polyoxyl 35 castor oil. KOLLIPHOR EL, formerly known as CREMOPHOR EL, is the registered trademark of BASF Corp. for its version of polyethoxylated castor oil. It is prepared by reacting 35 moles of ethylene oxide with each mole of castor oil. The resulting product is a mixture (CAS number 61791-12-6): the major component is the material in which the hydroxyl groups of the castor oil triglyceride have ethoxylated with ethylene oxide to form polyethylene glycol ethers. Minor components are the polyethylene glycol esters of ricinoleic acid, polyethylene glycols and polyethylene glycol ethers of glycerol. Polyoxyl 35 castor oil is also commercially available from Croda under the trade name of ETOCAS 35.

Other examples of suitable polyoxyethylene castor oil derivatives include polyoxyl 40 castor oil (commercially available from Sasol Performance Chemicals under the trade name MARLOWET 40, and commercially available from BASF under the trade name EMULGIN RO 40), polyoxyl 40 hydrogenated castor oil (commercially available from BASF under the trade name CREMOPHOR RH 40) and polyoxyl 60 hydrogenated castor oil (commercially available from BASF under the trade name CREMOPHOR RH 60).

The concentration of polyoxyethylene castor oil derivative in the present compositions may range from about 2% to about 60% by weight; in embodiments, from about 10% to about 50% by weight. Where the polyoxyethylene castor oil derivative employed is polyoxyl 35 castor oil, the concentration of polyoxyl 35 castor oil in the present compositions may range from about 5% to about 50% by weight; in embodiments, from about 15% to about 40% by weight.

As previously mentioned, the polyoxyethylene castor oil derivatives encapsulate nonpolar meibum-like materials to form nanoparticles that make up the presently disclosed clear ophthalmic compositions. While the following discussion focusses on cholesteryl/sterol esters and wax esters that are actual components of meibum, it should be understood that any nonpolar material that helps restore the oil phase or lipids of the tear film may be encapsulated by polyoxyethylene castor oil derivatives in accordance with the present disclosure.

Cholesteryl/sterol esters are formed via the esterization between cholesteryl/sterol and a fatty acid. The ester bond is between the carboxyl group of the fatty acid and the hydroxyl group of the cholesterol/sterol. Cholesteryl has the formula as C₂₇H₄₆O and molecular weight of 386.65. Other useful cholesteryl derivatives and sterols include lanosterol, 25-hydroxy-cholesterol, etc. Fatty acids suitable for esterification with the cholesterol include saturated and unsaturated acids with carbon chains ranging from C14 to C30. Non-limiting examples of suitable saturated fatty acids include myristic acid (C14), palmitic acid (C16), stearic acid (C18), arachidic acid (C20), behenic acid (C22), and lignoceric or tetracosanoic acid (C24). Useful unsaturated fatty acids include monounsaturated and multi-unsaturated acids. Non-limiting examples of suitable monounsaturated fatty acids include myristoleic acid (9 cis C14:1), palmitoleic acid (9 cis C16:1), oleic acid (9 cis C18:1), ricinoleic acid (9 cis 12 hydroxy C18:1), ricinelaidic acid (9 trans 12 hydroxy C18:1), gondolic acid (11 cis C20:1), erucic acid (13 cis C22:1), and nervonic acid (15 cis C24:1). Non-limiting examples of suitable multi-unsaturated fatty acids include linoleic acid (9 cis 12 cis C18:2), linoelaidic acid (9 trans 12 trans C18:2), alpha linolenic acid (9 cis 12 cis 15 cis C18:3), gamma linolenic acid (6 cis 9 cis 12 cis C18:3), homogamma linolenic acid (8 cis 11 cis 14 cis C20:3), arachidonic acid (5 cis 8 cis 11 cis 14 cis C20:4), eicosapentaenoic acid (EPA) (5 cis 8 cis 11 cis 14 cis 17 cis C20:5), and docosahexaenoic acid (DHA) (4 cis 7 cis 10 cis 13 cis 16 cis 19 cis C22:6).

In embodiments, the cholesteryl ester(s) are formed between cholesteryl and unsaturated fatty acids, and include cholesteryl palmitoleate (C16:1), cholesteryl oleate (C18:1), cholesteryl linoleate (C18:2), cholesteryl linolenate (C18:3), cholesteryl eicosapentaenoate (C20:5), cholesteryl docosapentaenoate (C22:5).

In embodiments, the cholesteryl ester(s) are formed between cholesteryl and multi-unsaturated fatty acids, and include, for example, cholesteryl linolenate (C18:3), which is commercially available from Nu-Chek-Prep. Multi-unsaturated cholesteryl esters have a lower melting point and are more likely to be liquid at the physiological temperature of 37° C.

The concentration of cholesteryl ester in the present composition may be from about 0.01% to about 1% by weight; in embodiments, from about 0.1% to about 0.5%.

Wax esters are formed via the esterization between one or more fatty alcohols and one or more fatty acids. The ester bond is between the carboxyl group of the fatty acid and the hydroxyl group of the fatty alcohol. Suitable fatty alcohols and fatty acids may be saturated or unsaturated (due to the presence of double bond(s) in the carbon chains) and may include carbon chains ranging from C14 to C30. Non-limiting examples of suitable saturated fatty alcohols/acids include myristic alcohol/acid (C14), palmitic alcohol/acid (C16), stearic alcohol/acid (C18), arachidic alcohol/acid (C20), behenic alcohol/acid (C22), and lignoceric alcohol/acid (C24). Suitable unsaturated fatty alcohols/acids include monounsaturated and multi-unsaturated alcohols/acids. Non-limiting examples of suitable monounsaturated fatty alcohols/acids include myristoleic alcohol/acid (9 cis C14:1), palmitoleic alcohol/acid (9 cis C16:1), oleic alcohol/acid (9 cis C18:1), ricinoleic alcohol/acid (9 cis 12 hydroxy C18:1), ricinelaidic alcohol/acid (9 trans 12 hydroxy C18:1), gondolic alcohol/acid (11 cis C20:1), erucic alcohol/acid (13 cis C22:1), and nervonic alcohol/acid (15 cis C24:1). Non-limiting examples of suitable multi-unsaturated fatty alcohols/acids include linoleic alcohol/acid (9 cis 12 cis C18:2), linoelaidic alcohol/acid (9 trans 12 trans C18:2), alpha linolenic alcohol/acid (ALA) (9 cis 12 cis 15 cis C18:3), gamma linolenic alcohol/acid (6 cis 9 cis 12 cis C18:3), homogamma linolenic alcohol/acid (8 cis 11 cis 14 cis C20:3), arachidonic alcohol/acid (5 cis 8 cis 11 cis 14 cis C20:4), eicosapentaenoic alcohol/acid (EPA) (5 cis 8 cis 11 cis 14 cis 17 cis C20:5), and docosahexaenoic alcohol/acid (DHA) (4 cis 7 cis 10 cis 13 cis 16 cis 19 cis C22:6).

In embodiments, the wax esters are formed between unsaturated fatty alcohols and unsaturated fatty acids, and include, for example, myristoleyl oleate, myristoleyl linoleate, myristoleyl linolenate, palmitoleyl oleate, palmitoleyl linoleate, palmitoleyl linolenate, oleyl oleate, oleyl linoleate, oleyl linolenate, linoleyl oleate, linoleyl linoleate, and/or linoleyl linolenate. Oleyl oleate is listed in USP and is commercially available from Lubrizol under the trade name SHERCEMOL OLO ester.

In embodiments, the wax esters are formed between monounsaturated fatty alcohols and multi-unsaturated fatty acids, and include, for example, oleyl linoleate, which is commercially available from Nu-Chek-Prep. Multi-unsaturated wax esters have a lower melting point and are more likely to be liquid at the room temperature of 25° C. and/or the physiological temperature of 37° C.

The concentration of wax ester in the present compositions may be from about 0.01% to about 1% by weight; in embodiments, from about 0.1% to about 0.5%.

In embodiments, one or more nonpolar lipid may be included in the present compositions. Any nonpolar lipid known to be suitable for ophthalmic compositions may be used. Suitable nonpolar lipids for use herein include triglycerides. Triglycerides are formed by reacting glycerine with a fatty acid. One molecule of glycerine can be esterized with three molecules of fatty acid. Triglycerides are the main components of the vegetable oil and fish oil (e.g. triglycerides of ω-3 fatty acids; EPA, DHA which are commercially available from Croda). In embodiments, the triglycerides are glyceryl trioleate and castor oil. Glyceryl trioleate is produced via the esterization of one molecule of glycerine with three molecules of oleic acid. The majority of castor oil is the esterization product between glycerine and ricinelaidic acid. Castor Oil is listed in USP and can be purchased from Spectrum Chemical.

In embodiments, the concentration of nonpolar lipids (e.g., triglycerides) in the present compositions may be up to about 0.5% by weight; in embodiments from about 0.05% to about 0.2%.

In embodiments, one or more polar lipid (e.g., a phospholipid), may be included in the present compositions. Phospholipids are formed between one molecule of glycerine and two molecules of fatty acid plus one molecule of phosphate group. The phosphate group can be modified with choline, ethanolamine, serine, etc. Any polar lipid known to be suitable for ophthalmic compositions may be used. Non-limiting examples of suitable phospholipids include soy lecithin (liquid lecithin) and 1,2-dimyristoyl-sn-glycero-3-phosphoglycerol, sodium salt (DMPG Na). Soy lecithin is liquid at room temperature and mostly a phosphatidylcholine (listed in USP/NF, and commercially available from Jedwards International). DMPG Na is a phosphatidylglycerol sodium salt, and is commercially available from Lipoid.

The concentration of polar lipids (e.g., phospholipids) used in the present compositions may be up to 0.1% by weight; in embodiments, from about 0.02% to about 0.05%.

In embodiments, one or more buffering agents may be included in the present compositions. Any buffering agent known to be suitable for ophthalmic compositions may be used. In embodiments, the buffering agents may include one or more of boric acid, sodium borate, sodium hydroxide, tromethamine, amino methyl propanol, or combinations thereof. Suitable combinations of buffering agents useful in the present compositions include: a) boric acid and sodium borate, b) boric acid and sodium hydroxide, c) boric acid and tromethamine, d) boric acid and amino methyl propanol, or combinations thereof. In embodiments, the buffering agents employed are boric acid and tromethamine.

The total concentration of buffering agents used in the present compositions may be up to 5%, depending on the buffering agent or combination of buffering agents chosen. In embodiments, the buffering agent includes boric acid in an amount from about 0.1% to about 2% by weight; in embodiments, from about 0.5% to about 1%. In embodiments, the buffering agent includes tromethamine, alone or in combination with boric acid. When present, the amount of tromethamine in the present compositions may be from about 0.05% to about 1% by weight; in embodiments, tromethamine is present in an amount from about 0.1% to about 0.5%.

In embodiments, one or more chelating agents may be included in the present compositions. Any chelating agent known to be suitable for ophthalmic compositions may be used. A non-limiting example of a suitable chelating agent is edetate disodium (EDTA). The total concentration of chelating agents used in the present compositions may be up to 2%. In embodiments where EDTA is used as the chelating agent, EDTA may be present in the composition in an amount from about 0.01% to about 0.1% by weight.

In embodiments, one or more lubricant/demulcent agents may be included in the present compositions. Any lubricant/demulcent agent known to be suitable for ophthalmic compositions may be used. Non-limiting examples of suitable lubricant/demulcent agents include glycerine, propylene glycol, sorbitol, polyethylene glycol 400 (PEG 400), and polysorbate 80. It should, of course, be understood that combinations of these lubricant/demulcent agents may be included in the present compositions. The total concentration of lubricant/demulcent agents used in the present compositions may be up to 5%. In embodiments where glycerine is used as the lubricant/demulcent agent alone or in combinations with other lubricant/demulcent agents, glycerine may be present in the present compositions in an amount from about 0.1% to about 1% by weight. In embodiments where propylene glycol is used as the lubricant/demulcent agent alone or in combinations with other lubricant/demulcent agents, propylene glycol may be present in the present compositions in an amount from about 0.1% to about 0.5% by weight. In embodiments where sorbitol is used as the lubricant/demulcent agent alone or in combinations with other lubricant/demulcent agents, sorbitol may be present in the present compositions in an amount from about 0.1% to about 0.5% by weight. In embodiments where PEG 400 is used as the lubricant/demulcent agent alone or in combinations with other lubricant/demulcent agents, PEG 400 may be present in the present compositions in an amount from about 0.1% to about 0.5% by weight. In embodiments where polysorbate 80 is used as the lubricant/demulcent agent alone or in combinations with other lubricant/demulcent agents, polysorbate 80 may be present in the present compositions in an amount from about 0.1% to about 1.0% by weight.

In embodiments, one or more gelling agents may be included in the present compositions. Any gelling agent known to be suitable for ophthalmic compositions may be used. Non-limiting examples of suitable gelling agents include, for example, ionic and nonionic polysaccharides, such as, for example, guar gum, gellan gum, xantham gum, and the like. In embodiments, gellan gum is used as the gelling agent. Gellan gum is commercially available, for example, from Kelco under the trade name GELRITE. GELRITE gellan gum interestingly starts gelling when exposed to metal ions present in the tear film. The total concentration of gelling agents used in the present compositions may be up to 2%. In embodiments where gellan gum is used as the gelling agent alone or in combinations with other gelling agents, gellan gum may be present in the present compositions in an amount from about 0.001% to about 0.5% by weight; in embodiments, from about 0.002% to about 0.1%.

In embodiments, one or more thickening agents may be included in the present compositions. Any thickening agent known to be suitable for ophthalmic compositions may be used. Non-limiting examples of suitable thickening agents include, for example, hypromellose or hydroxypropyl methylcellulose (HPMC), hydroxyethylcellulose (HEC), carboxymethylcellulose sodium (CMC Na), polyvinyl alcohol (PVA), povidone, and sodium hyaluronate. In embodiments, HEC is used as the thickening agent. HEC is commercially available, for example, from Hercules under the trade name NATROSOL. Among the useful HEC products available from Hercules is NATROSOL 250M. The total concentration of thickening agents used in the present compositions may be up to 2%. In embodiments where HEC is used as the thickening agent alone or in combinations with other thickening agents, HEC may be present in the present compositions in an amount from about 0.1% to about 0.5% by weight.

In embodiments, a preservative system may be included in the present compositions. Any preservative system known to be suitable for ophthalmic compositions may be used. Non-limiting examples of suitable preservatives include, for example, benzalkonium chloride, stabilized chlorine peroxide complexes, polyquaternary ammonium compounds such as polyquaternium-1 (POLYQUAD), polyquaternium-42 (BUSAN 1507, Buckman Laboratories), and polyhexamethylene biguanide (PHMB or polyhexanide), chlorobutanol, thimerosal, sorbic acid, chlorhexidine gluconate. In embodiments, polyhexamethylene biguanide (PHMB or polyhexanide) is used as the preservative. The total concentration of preservatives used in the present compositions may be up to 10 ppm. In embodiments where PHMB is used as the preservative alone or in combinations with other preservatives, PHMB may be used in the present compositions in an amount from about 1 ppm to about 4 ppm. In embodiments, the present compositions may be prepared as preservative-free compositions.

Microemulsion systems in accordance with the present disclosure can be further utilized to carry pharmaceutically active agents in the nanoparticle capsules, including glaucoma therapeutics, pain relievers, anti-inflammatory compounds, anti-allergy medications, and anti-microbials. Non-limiting examples of pharmaceutically active agents that may be included in the present ophthalmic compositions include Naphazoline HCl, Tetrahydrozoline HCl, Cyclosporine, Timolol, Dorzolamide, Pilocarpine, Brimonidine Tartrate, Olopatadine, Epinastine, Betaxolol, Pheniramine Maleate, Lotepredenol Etabonate, Ciprofloxacin, Ofloxacin, Gentamicin, Flurbiprofen, Gramicidin, Erythromycin, Levofloxacin, Moxifloxacin, Neomycin, Polymyxin B, Sodium Sulfacetamide, Tobramycin, Bacitracin, Dexamethasone, Flurometholone, Hydrocortisone, Prednisolone, Levalbuterol Hydrochloride, Trifluridine, Naproxen, Diclofenac, Bromfenac, Ketotifen Fumarate, Travoprost, Latanoprost, Bimatoprost, Tropicamide, Phenylephrine, Tetracaine, Proparacaine, Benoxinate, and Lidocaine. In embodiments, the clear microemulsion compositions in accordance with the present disclosure contain sufficient hydrophobic groups to serve as a vehicle to solubilize/deliver some poorly water-soluble pharmaceutically active drugs, e.g. cyclosporine, erythromycin, hydrocortisone, bacitracin, prostaglandins, etc.

The pharmaceutically active agent(s) may be incorporated into the present compositions in a therapeutically effective amount. The term “effective amount” or “therapeutically effective amount” is an amount sufficient to affect a therapeutically beneficial or therapeutically desired result. A therapeutically effective amount can be administered in one or more administrations, applications or dosages. In embodiments, pharmaceutically active agent(s) may be incorporated into the present compositions in an amount up to about 2.0% by weight, in embodiments from about 0.005 to about 1.0%.

The composition, whether prepared with or without any preservative, may be sterilized. In embodiments, the composition may be sterilized by heating the composition to a temperature that eliminates any bacteria or other living microorganisms. In other embodiments, the composition may be sterilized by passing it through a membrane filter having a sufficiently small pore size to eliminate any bacteria or other living microorganisms. In embodiments, the present composition is passed through a 0.2 μm membrane filter.

The following Table 1 provides ranges of ingredients for illustrative embodiments of compositions in accordance with this disclosure.

TABLE 1

| mg/mL | Ingredient | Percentage (%) |
|---|---|---|
| Qs. 1.0 mL | Water | Qs. 100% |
| 0-11.20 | Boric Acid | 0%-1.12% |
| 0-6.50 | Tromethamine | 0%-0.65% |
| 1.00-5.00 | PEG 400, Usp | 0.1%-0.5% |
| 1.00-5.00 | Propylene Glycol | 0.1%-0.5% |
| 0.10-1.00 | Oleyl Linoleate | 0.01%-0.1% |
| 0.10-1.00 | Cholesteryl Linolenate | 0.01%-0.1% |
| 1.00-10.00 | Sorbitol, 70% Solution | 0.1%-1.0% |
| 1.00-10.00 | Polysorbate 80 | 0.1%-1.0% |
| 1.00-10.00 | Glycerine | 0.1%-1.0% |
| 50.00-400.00 | Polyoxyl 35 Castor Oil | 5%-40% |
| 1.00-5.00 | PHMB, 0.1% Solution | 0.0001%-0.0005% |

Desired Physico-chemical properties

| | |
|---|---|
| Appearance | Clear, colorless to slightly yellowish solution |
| pH | 6.0 to 8.0 |
| Osmolality | 250 to 400 mOsm/kg |

The present compositions may be prepared using any technique within the purview of those skilled in the art. In embodiments, the compositions are prepared by: first preparing a mixture of the aqueous components (Part A) with heating if needed; preparing a mixture of the remaining, mostly hydrophobic components (Part B) with heating if needed; combining Parts A and B with continued mixing and heating if needed. Mixing should be continued until a clear, colorless, or slightly yellowish solution forms. In some cases, mixing may need to be continued for up to 18 hours or more.

In embodiments, Part A includes water, lubricant/demulcent agent(s), buffering agent(s), and preservative(s). In embodiments, the ingredients for Part A include: boric acid, tromethamine, sorbitol, polysorbate 80, glycerine, PEG 400, propylene glycol, polyhexamethylene biguanide, and water.

In embodiments, Part B includes nonpolar meibum-like materials (such as, for example, cholesteryl/sterol ester(s) and wax ester(s)), polyoxyethylene castor oil derivative(s), nonpolar lipid(s), and polar lipid(s) (e.g., phospholipids). In embodiments, the ingredients for Part B include: polyoxyl 35 castor oil, cholesteryl linolenate, oleyl linoleate, glyceryl trioleate, castor oil, liquid lecithin, and DMPG-Na.

In embodiments, the appearance of the present composition may be clear to colorless. In other embodiments, the appearance of the present composition may be pale yellow. In embodiments, the appearance of the present composition is neither cloudy nor milky.

In embodiments, the size of nanoparticles in the microemulsion system of the present disclosure may be from about 2 nm to about 100 nm, in embodiments from about 5 to about 50 nm, as observed utilizing a submicron particle sizer (available, e.g., from Particle Sizing Systems, Santa Barbara, Calif. now part of Entegris Inc., Billerica, Mass., USA).

In embodiments, the tonicity of an eye drop in accordance with embodiments of the present disclosure may be from about 250 mOsm/kg to about 400 mOsm/kg; in embodiments from about 280 mOsm/kg to about 330 mOsm/kg.

In embodiments, the pH of the present compositions may be from about 6.0 to about 8.0.

In embodiments, the viscosity of an eye drop in accordance with embodiments of the present disclosure may be from 5 cps to 50 cps.

In embodiments, the viscosity of an ointment/gel in accordance with embodiments of the present disclosure may be from 100,000 cps to 800,000 cps.

In embodiments, the specific gravity of the present compositions may be 1.000 to 1.020.

### EXAMPLES

The following examples are presented to illustrate specific aspects of compositions in accordance with the present disclosure and their properties. They are different composition systems to reflect various aspects of non-limiting, illustrative examples of nanoparticle microemulsions of nonpolar lipids and/or active drugs.

### Example 1

Meibum and meibum-like materials (cholesteryl ester and wax ester) can be formulated inside nanoparticles in compositions in accordance with the present disclosure to produce eye drop products capable of supplying meibum materials or the like directly into the eyes of a patient suffering from dry eye or MGD where lipid layer materials are deficient and/or compromised. Such eye drops address deficiencies of both the lipid layer and aqueous layer.

Illustrative formulations of eye drop preparations containing cholesteryl ester and wax ester in accordance with embodiments of the present disclosure are shown in Table 2 below.

TABLE 2
Eye Drop Compositions

| | Amount (weight by volume %) | | | | |
|---|---|---|---|---|---|
| Ingredient | a | b | c | d | e |

Part A

| | | | | | |
|---|---|---|---|---|---|
| Boric Acid | 0.84 | 0.56 | 0.56 | 0.56 | 0.56 |
| Tromethamine | 0.488 | 0.30 | 0.30 | 0.25 | 0.15 |
| Sorbitol | 0.175 | 0.07 | 0.175 | 0.07 | 0.07 |
| Polysorbate 80 | 0.50 | 0.25 | 0.50 | 0.25 | 0.25 |
| Glycerine | 0.25 | 0.10 | 0.25 | 0.10 | 0.10 |
| PEG 400 | 0.40 | 0.40 | 0.40 | 0.40 | 0.40 |
| Propylene Glycol | 0.30 | 0.30 | 0.30 | 0.30 | 0.30 |
| Polyhexamethylene | 0.0002 | 0.0002 | 0.0002 | 0.0002 | 0.0002 |
| Biguanide (PHMB) | | | | | |
| Water | Qs 100% | Qs 100% | Qs 100% | Qs 100% | Qs 100% |

Part B

| | | | | | |
|---|---|---|---|---|---|
| Polyoxyl 35 Castor Oil | 25.00 | 25.00 | 20.00 | 22.00 | 22.00 |
| Cholesteryl Linolenate | 0.25 | 0.25 | 0.15 | 0.15 | 0.15 |
| Oleyl Linoleate | 0.25 | 0.25 | 0.15 | 0.15 | 0.15 |
| Glyceryl Trioleate | 0.05 | 0.05 | 0.05 | 0.05 | 0.05 |
| Castor Oil | 0.10 | 0.10 | 0.05 | 0.05 | 0.05 |
| Liquid Lecithin | 0.05 | 0.05 | 0.05 | 0.025 | 0.025 |
| DMPG-Na | 0.01 | 0.01 | 0.01 | 0.01 | 0.01 |

Physico-chemical properties

| | | | | | |
|---|---|---|---|---|---|
| Appearance | clear | clear | clear | clear | clear |
| pH | 7.40 | 7.53 | 7.36 | 7.57 | 7.36 |
| Osmolality (mOsm/kg) | 633 | 437 | 343 | 330 | 313 |
| Viscosity (cps) | 26 | 22 | 7.1 | 8.2 | 8.3 |
| Mean particle size (nm) | 22.6 | 20.4 | 14.3 | 14.0 | 14.0 |

Part A of the eye drop formulations in Table 2 is aqueous and the ingredients thereof can be mixed together with a propeller mixer at >70° C. Part B of the eye drop formulations in Table 2 is mostly hydrophobic and the ingredients thereof can be mixed together with a propeller mixer at >70° C. Thereafter, Part B is transferred into Part A with continued mixing while keeping both phases at >70° C. The combined preparation yields a clear, colorless to slightly yellowish solution. The batch can be sterilized by passing the composition through a 0.2 μm filtration membrane at >50° C. Containing no preservative, such a composition is “preservative-free”.

Desired physico-chemical properties of eye drop compositions to treat MGD include:

pH in the range of 7.0 to 8.0.

Osmolality in the range of 280 mOsm/kg to 330 mOsm/kg.

Viscosity in the range of 5 to 50 cps

Particle size in the range of 5 to 100 nm

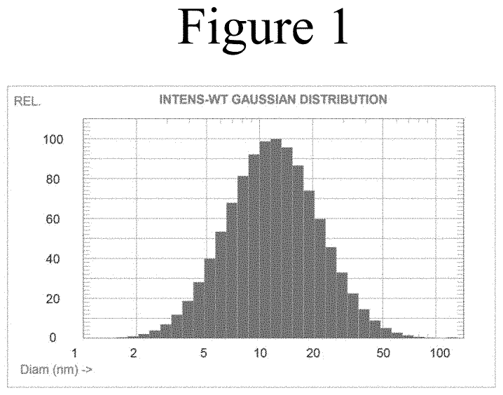

FIG. 1 shows a Gaussian distribution of an eye drop composition in accordance with an embodiment of the present disclosure to treat MGD (Example 1, composition c). The mean size of the nanoparticles is 14.3 nm and the size range of the nanoparticles is from 2 nm to 80 nm.

Eye drop compositions in accordance with the present disclosure to treat MGD were diluted into water for injection and a balanced salt solution (BSS) to simulate the instillation into human tears (e.g., 30 mL of eye drop product into 10 mL simulated tears). No separation of the nonpolar lipids from the aqueous phase was observed, and the mean particle size remained less than 20 nm.

Eye drop compositions in accordance with the present disclosure to treat MGD contain essential lipids found in human meibum and the tear film lipid layer, such as cholesteryl ester, wax ester, triglyceride, phospholipid, and remain to be clear with nano-packaged essential lipids in the desired particle size range. Such products will not have any blurring effect upon application to the eyes of a patient, which is a general complaint for many of the commercial dual-action eye drops currently marketed that include either mineral oil and/or castor oil.

### Example 2

Compositions in accordance with the present disclosure containing meibum-like materials can be formulated to demonstrate significant viscosity (e.g. >100,000 cps) while remaining a clear gel/ointment at room temperature. Such compositions are aqueous-based formulations rather than the traditional ophthalmic ointments made of white petrolatum and a mineral oil base.

Various ophthalmic ointment preparations containing nonpolar mineral oil, cholesteryl ester, and wax ester in accordance with the present disclosure are shown in Table 3 below.

TABLE 3
Ointment Compositions

| | Amount (weight by volume %) | | | |
|---|---|---|---|---|
| Ingredient | a | b | c | d |

Part A

| | | | | |
|---|---|---|---|---|
| Boric Acid | 0.56 | 0.56 | 0.56 | 0.56 |
| Tromethamine | 0.30 | 0.30 | 0.30 | 0.30 |
| Sorbitol | 0.35 | 0.35 | 0.35 | 0.35 |
| Polysorbate 80 | 0.50 | 0.50 | 0.50 | 0.50 |
| Glycerine | 0.50 | 0.50 | 0.50 | 0.50 |
| PEG 400 | 0.40 | 0.40 | 0.40 | 0.40 |
| Propylene Glycol | 0.30 | 0.30 | 0.30 | 0.30 |
| Polyhexamethylene | 0.0002 | 0.0002 | 0.0002 | 0.0002 |
| Biguanide (PHMB) | | | | |
| Water | Qs 100% | Qs 100% | Qs 100% | Qs 100% |

Part B

| | | | | |
|---|---|---|---|---|
| Polyoxyl 35 Castor | 40.00 | 40.00 | 40.00 | 40.00 |
| Oil | | | | |
| Light Mineral Oil | 3.00 | 2.00 | 1.00 | 1.00 |
| Cholesteryl | — | — | — | 0.15 |
| Linolenate | | | | |
| Oleyl Oleate | 0.50 | — | — | — |
| Oleyl Linoleate | 0.20 | — | — | 0.15 |
| Glyceryl Trioleate | 0.10 | 0.10 | 0.10 | 0.10 |
| Castor Oil | 0.20 | 0.10 | 0.10 | 0.10 |
| Liquid Lecithin | 0.05 | 0.05 | 0.05 | 0.05 |
| DMPG-Na | 0.02 | 0.02 | 0.02 | 0.02 |
| Lanolin Oil | 0.05 | — | — | — |

Physico-chemical properties

| | | | | |
|---|---|---|---|---|
| Appearance | clear | clear | clear | clear |
| pH | 7.53 | 7.30 | 7.29 | 7.27 |
| Osmolality | 660 | 484 | 492 | 524 |
| (mOsm/kg)* | | | | |
| Viscosity (cps) | >10⁶ | >10⁶ | 350,000 | >10⁶ |
| Mean particle size | 12.5 | 13.9 | 20.9 | 16.2 |
| (nm) | | | | |

*reading was obtained by performing 1:2 dilution in water

Part A of the ointment compositions of Table 3 is aqueous and includes ingredients that can be mixed together with a propeller mixer at >70° C. Part B of the ointment compositions of Table 3 is mostly hydrophobic and includes ingredients that can be mixed together with a propeller mixer at >70° C. Thereafter, Part B is transferred into Part A with mixing while keeping both phases at >70° C. The combined preparation yields a clear, colorless to slightly yellowish gel/solution. The batch can be sterilized by passing the composition through a 0.2 μm filtration membrane at >50° C.

Desired physico-chemical properties of ointment compositions in accordance with the present disclosure include:

pH in the range of 7.0 to 8.0.

Osmolality in the range of 250 mOsm/kg to 500 mOsm/kg.

Viscosity in the range of 100,000 to 800,000 cps

Particle size in the range of 5 to 100 nm

The present ointment preparations were diluted into water for injection and balanced salt solution (BSS) to simulate the instillation into human tears (e.g., 30 mL of the ointment product into 10 mL simulated tears). No separation of the nonpolar lipids from the aqueous phase was observed and the mean particle size remained to be less than 20 nm.

Ointment compositions in accordance with embodiments of the present disclosure contain lipids found in human meibum and the tear film lipid layer, such as cholesteryl ester, wax ester, triglyceride, and phospholipid. The ointment is an aqueous-based product and demonstrates better compatibility with tear film than current commercially available ophthalmic oleaginous ointments.

### Example 3

Shown below are examples of illustrative microemulsion compositions containing a pharmaceutically active agent; namely, cyclosporine (Table 4).

TABLE 4
Microemulsion Containing Cyclosporine

| | Amount (weight by volume %) | | | |
|---|---|---|---|---|
| Ingredient | a | b | c | d |

Part A

| | | | | |
|---|---|---|---|---|
| Boric Acid | 1.12 | 1.12 | 1.12 | 1.12 |
| Tromethamine | 0.65 | 0.65 | 0.65 | 0.65 |
| Sorbitol | 0.70 | 0.70 | 0.70 | 0.35 |
| PEG 400 | 0.40 | — | — | 0.40 |
| Propylene Glycol | 0.30 | 0.30 | 0.30 | 0.30 |
| Edetate Disodium | — | — | 0.10 | — |
| Water | Qs 100% | Qs 100% | Qs 100% | Qs 100% |
| Benzalkonium Chloride | 0.01 | — | — | — |
| Polyhexamethylene | — | 0.00015 | 0.00015 | — |
| Biguanide (PHMB) | | | | |

Part B

| | | | | |
|---|---|---|---|---|
| Cyclosporine | 0.05 | 0.05 | 0.10 | 0.05 |
| Castor Oil | 1.00 | 0.625 | 0.625 | 0.30 |
| Polyoxyl 35 Castor Oil | 5.00 | 3.125 | 3.125 | 1.50 |
| Appearance | clear | clear | clear | clear |
| pH | 7.00 | 6.90 | 6.87 | 7.22 |
| Osmolality (mOsm/kg.) | 288 | 280 | 275 | 303 |
| Mean particle size (nm) | 20.9 | 22.3 | 20.9 | 24.6 |

The compositions of Table 4 contain 0.05% and 0.1% cyclosporine and include nanoparticles having a mean particle size of less than 25 nm, and therefore are clear compositions. Preparations with larger particles (e.g., mean particle size>30 nm) that demonstrated haziness/cloudiness are not shown here.

Part A was prepared by adding the ingredients into water, one at a time until dissolved while mixing. Part B was prepared by first adding cyclosporine into castor oil, mixing until dissolved, and then adding polyoxyl 35 castor oil. After mixing each phase of Part A and Part B homogeneously, Part B was transferred into Part A. The main batch can be sterilized by autoclaving at 121° C. for 30-45 minutes. The size of nanoparticles was found to remain the same before and after autoclaving. The main batch can alternatively be sterilized via passing the composition through a 0.2 μm filtration membrane. In such instances, the compositions can be identified as “preservative-free”.

Desired physico-chemical properties of microemulsions containing cyclosporine in accordance with the present disclosure include: pH in the range of 6.5 to 7.5; osmolality in the range of 270 mOsm/kg to 330 mOsm/kg; and particle size in the range of 5 nm to 100 nm.

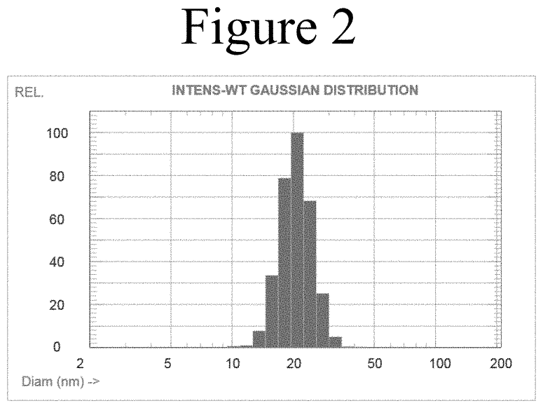

FIG. 2 shows a Gaussian distribution of an ophthalmic microemulsion in accordance with the present disclosure containing 0.1% (w/v) cyclosporine (Example 3, composition c). The mean size of the nanoparticles is 20.9 nm and the size range of the nanoparticles is from 5 nm to 50 nm.

Dry eye disease with immune medicated keratoconjunctivitis sicca (KCS) may be treated using the above exemplary compositions containing 0.1% cyclosporine (e.g., Example 3, composition d) in accordance with the present disclosure. The clear appearance of the present cyclosporine-containing ophthalmic solution is desirable compared to products currently marketed, which are white milky emulsion and have a mean particle size of about >500 nm. A clear, much smaller particle size 0.1% cyclosporine ophthalmic solution in accordance with the present disclosure results in no blurring of vision and improved bioavailability of the active drug.

### Example 4

The safety of microemulsion compositions in accordance with embodiments of the present disclosure containing, e.g., 5%-40% of polyoxyl 35 castor oil was evaluated via Draize test and MTT assay. Microemulsion compositions in accordance with embodiments of the present disclosure with 8%, 15%, 25%, and 50% of polyoxyl 35 castor oil were submitted to Pacific Biolabs for an ocular irritation study—Draize rabbit eye test. The study design and results are summarized below in Table 5:

TABLE 5
Ocular Irritation Tests

| Animal | | | | Dose |
|---|---|---|---|---|
| Number (n) | Treatment | Eyes | Dose (mL) | schedule |
| 3 | Test | Right (Test) | 0.1 | Once a Day |
| New Zealand | Control | Left (Control) | 0.1 | Once a Day |
| White Rabbits | | | | |

| Concentration of | Dose | | |
|---|---|---|---|
| Polyoxyl 35 Castor Oil | schedule | Scoring | Results* |
| 8% | Once 0.1 mL | 1 hr ± 6 min | Not positive |
| | | 24 ± 2 hrs | irritation |
| | | 48 ± 2 hrs | response |
| 15% | Once 0.1 mL | 72 ± 2 hrs | Not positive |
| | | | irritation |
| | | | response |
| 25% | Once 0.1 mL | | Not positive |
| | | | irritation |
| | | | response |
| 50% | Once 0.1 mL | | Not positive |
| | | | irritation |
| | | | response |

The Draize test was conducted by applying the stated dosage of the microemulsion compositions on three individual rabbits and observing for any indication of irritation. Scores were recorded at 1 hour, 24 hours, 48 hours, and 72 hours after dosing. No positive irritation response was found for any of the studied compositions.

A microemulsion preparation in accordance with an embodiment of the present disclosure with 40% polyoxyl 35 castor oil was also submitted to Pacific Biolabs for a customized ocular irritation study—Draize rabbit eye test with an additional challenge. The study design and results are summarized below in Table 6.

TABLE 6
Challenged Ocular Irritation Tests

| Animal | | | | Dose |
|---|---|---|---|---|
| Number (n) | Treatment | Eyes | Dose | schedule |
| 3 | Test | Right | 60 μL | Twice a Day |
| New Zealand | | (Test) | | (approximately |
| White Rabbits | | | | 6 to 8 hours |
| | | | | apart) for 10 |
| | | | | consecutive days |
| | Control | Left | 60 μL | Twice a Day |
| | | (Control) | | (approximately |
| | | | | 6 to 8 hours |
| | | | | apart) for 10 |
| | | | | consecutive days |

| Concentration of | Dose | | |
|---|---|---|---|
| polyoxyl 35 castor oil | schedule | Scoring | Results* |
| 40% | Day 1, two | 1 hr ± 6 | No positive |
| | drops, twice | min post | irritation response |
| | Day 2, two | dosing | No positive |
| | drops, twice | Prior to | irritation response |
| | Day 3, two | next | No positive |
| | drops, twice | dosing | irritation response |
| | Day 4, two | | No positive |
| | drops, twice | | irritation response |
| | Day 5, two | | No positive |
| | drops, twice | | irritation response |
| | Day 6, two | | No positive |
| | drops, twice | | irritation response |
| | Day 7, two | | No positive |
| | drops, twice | | irritation response |
| | Day 8, two | | No positive |
| | drops, twice | | irritation response |
| | Day 9, two | | No positive |
| | drops, twice | | irritation response |
| | Day 10, two | | No positive |
| | drops, twice | | irritation response |

The Draize test was conducted by performing the repeated dosage of the microemulsion containing 40% polyoxyl 35 castor oil in accordance with an embodiment of the present disclosure on three individual rabbits for ten (10) consecutive days, and observing for any indication of irritation. Scores were recorded at each study day prior to next dosing. No positive irritation response was found for the microemulsion containing 40% polyoxyl 35 castor oi.

Another Ocular Irritation Study was done by exposing a microemulsion containing polyoxyl 35 castor oil in accordance with an embodiment of the present disclosure onto normal human keratinocytes/stratified squamous epithelium which is similar to that found in the cornea epithelium of the human eye (EPIOCULAR model), and then checking tissue viability with MTT assay for cytoxicity after incubation in comparison with a water control (Cyprotex). The study design and results are summarized below in Table 7.

TABLE 7
Ocular Irritation Tests - MTT Assays

| | Mean Tissue* | Irritation Response |
|---|---|---|
| Sample ID | Viability | (Irritant if ≤60%) |
| Methyl Acetate | 31.1% | Positive |
| (Positive Control) | | |
| Water | 100.0% | Negative |
| (Negative Control) | | |

Concentration of polyoxyl 35 castor oil

| | | |
|---|---|---|
| 25% microemulsion | 98.4% | Not Irritant |
| 40% microemulsion | 102.4% | Not Irritant |

As the foregoing data shows, microemulsion formulations containing 5%-40% polyoxyl 35 castor oil in accordance with embodiments of the present disclosure demonstrated “nonirritant response” in the ocular irritation (OIT) studies, including both Draize test and the MTT assay for cytotoxicity. Microemulsion formulations containing 40% polyoxyl 35 castor oil in accordance with embodiments of the present disclosure ware further challenged with a customized Draize test under repeated dosage conditions for up to ten (10) days. Those studies consistently suggest that the present compositions with 5%-40% polyoxyl 35 castor oil were not irritating towards the New Zealand White rabbit's eyes in Draize test and/or the tissues/normal human keratinocytes utilized in the MTT assay for cytotoxicity. Accordingly, microemulsions in accordance with embodiments of the present disclosure containing polyoxyl 35 castor oil in the range of 5%-40% are shown to be safe to use as ophthalmic preparations.

Various modifications of the disclosed concepts and embodiments, in addition to those described herein, will be apparent to those skilled in the art from the foregoing description. Various modifications, substitutions, and variations can be made to the disclosed embodiments without departing from the essential characteristics of the present disclosure. As such, any modifications, variations, or substitutions, which may occur to those skills in the art, should be considered to be within the scope of the present disclosure.

What is claimed is:

1. An artificial tear formulation comprising;
nanoparticles having one or more nonpolar meibum-like material encapsulated by at least one polyoxyethylene castor oil derivative,
wherein the formulation exhibits a clear appearance.

2. The formulation of claim 1 wherein the nanoparticles have a mean size of from about 5 nm to about 100 nm.

3. The formulation of claim 1 having a viscosity from about 1 cps to about 1000 cps.

4. The formulation of claim 1 wherein the at least one meibum-like material includes at least one cholesteryl ester or at least one wax ester.

5. The formulation of claim 4 wherein the at least one meibum-like material includes cholesteryl linolenate.

6. The formulation of claim 4 wherein the at least one meibum-like material includes oleyl linoleate.

7. The formulation of claim 1 wherein the at least one polyoxyethylene castor oil derivative includes polyoxyl 35 castor oil.

8. The formulation of claim 7 wherein polyoxyl 35 castor oil is present at a concentration of from about 1% to about 50%.

9. The formulation of claim 1 wherein the formulation has a pH of 6.0-8.0, and an osmolality of from about 250 mOsm/kg to about 400 mOsm/kg.

10. The formulation of claim 1 further comprising one or more of a buffer, a lubricant/demulcent agent, or a preservative.

11. The formulation of claim 1 wherein the nanoparticles have a mean size of from about 10 nm to about 30 nm and the artificial tear formulation has a viscosity from about 100,000 cps to more than about 1,000,000 cps.

12. A method comprising
administering an artificial tear formulation to a patient experiencing a dry eye condition, the formulation exhibiting a clear appearance and having nanoparticles with one or more nonpolar meibum-like material encapsulated by at least one polyoxyethylene castor oil derivative.

13. An artificial tear formulation comprising:
nanoparticles having one or more pharmaceutically acceptable active agent encapsulated by at least one polyoxyethylene castor oil derivative,
wherein the formulation exhibits a clear appearance.

14. The formulation of claim 12 wherein the one or more pharmaceutically acceptable active agents is selected from Naphazoline HCl, Tetrahydrozoline HCl, Cyclosporine, Timolol, Dorzolamide, Pilocarpine, Brimonidine Tartrate, Olopatadine, Epinastine, Betaxolol, Pheniramine Maleate, Lotepredenol Etabonate, Ciprofloxacin, Ofloxacin, Gentamicin, Flurbiprofen, Gramicidin, Erythromycin, Levofloxacin, Moxifloxacin, Neomycin, Polymyxin B, Sodium Sulfacetamide, Tobramycin, Bacitracin, Dexamethasone, Flurometholone, Hydrocortisone, Prednisolone, Levalbuterol Hydrochloride, Trifluridine, Naproxen, Diclofenac, Bromfenac, Ketotifen Fumarate, Travoprost, Latanoprost, Bimatoprost, Tropicamide, Phenylephrine, Tetracaine, Proparacaine, Benoxinate, Lidocaine, or combinations thereof.

15. The formulation of claim 12 wherein the nanoparticles have a mean size of from about 5 nm to about 100 nm.

16. The formulation of claim 12 having a viscosity from about 1 cps to about 1000 cps.

17. The formulation of claim 12 wherein the at least one polyoxyethylene castor oil derivative includes polyoxyl 35 castor oil.

18. The formulation of claim 17 wherein polyoxyl 35 castor oil is present at a concentration of from about 1% to about 50%.

19. The formulation of claim 12 wherein the formulation has a pH of 6.0-8.0, and an osmolality of from about 250 mOsm/kg to about 400 mOsm/kg.

20. The formulation of claim 12 further comprising one or more of a buffer, a lubricant/demulcent agent, or a preservative.

21. An ophthalmic ointment preparation comprising;
nanoparticles having one or more nonpolar meibum-like material encapsulated by at least one polyoxyethylene castor oil derivative,
wherein the formulation exhibits a clear appearance and a viscosity>100,000 cps.

22. The ophthalmic ointment preparation of claim 21 wherein the nanoparticles have a mean size of from about 12 nm to about 20 nm.

23. The ophthalmic ointment preparation of claim 21 having a viscosity from about 350,000 cps to >10⁶ cps.

24. The ophthalmic ointment preparation of claim 21 wherein the at least one meibum-like material includes at least one cholesteryl ester.

25. The ophthalmic ointment preparation of claim 21 wherein the at least one polyoxyethylene castor oil derivative includes polyoxyl 35 castor oil.

26. The ophthalmic ointment preparation of claim 21 wherein the preparation has a pH of 6.0-8.0, and an osmolality of from about 475 mOsm/kg to about 660 mOsm/kg.
